(19) United States
(12) Patent Application Publication
(10) Pub. No.: US 2023/0346678 A1
(43) Pub. Date: Nov. 2, 2023

(54) TOPICAL COMPOSITIONS CONTAINING VITAMIN C

(71) Applicant: Johnson & Johnson Consumer Inc., Skillman, NJ (US)

(72) Inventors: Marisa Dufort, Hillsborough, NJ (US); Nathaniel Grande, Hillsborough, NJ (US); Ritamarie Guerrero, Hillsborough, NJ (US)

(21) Appl. No.: 18/139,476

(22) Filed: Apr. 26, 2023

Related U.S. Application Data
(60) Provisional application No. 63/335,884, filed on Apr. 28, 2022.

(51) Int. Cl. A61K 8/67; A61K 8/49; A61Q 19/00

(52) U.S. Cl. CPC A61K 8/676; A61K 8/498; A61Q 19/00; A61K 2800/80

(57) ABSTRACT

The present invention provides topical compositions comprising a combination ascorbic acid, epigallocatechin gallate, water and 1,3-propanediol.

### CROSS-REFERENCE TO RELATED APPLICATIONS

This application claims the benefit of U.S. provisional application 63/335,884 filed on Apr. 28, 2022, the complete disclosure of which is hereby incorporated herein by reference for all purposes.

### FIELD OF THE INVENTION

The present invention provides topical compositions comprising a combination ascorbic acid, epigallocatechin gallate, water and 1,3-propanediol.

### BACKGROUND OF THE INVENTION

Ascorbic acid (Vitamin C) is widely used in skincare for its antioxidant and skin brightening properties. Anhydrous ascorbic acid formulations are not as effective in delivering ascorbic acid as aqueous systems. However, the stability of ascorbic acid in an aqueous vehicle is known to be challenging as water increases the oxidation kinetics for ascorbic acid.

Applicants have now discovered improved topical compositions, preferably in the form of serums, containing high levels of ascorbic acid stabilized in a unique aqueous vehicle containing 1,3-propanediol. The compositions also contain epigallocatechin gallate (EGCG), a polyphenol found in green tea also having powerful antioxidant properties. The combination of ascorbic acid and EGCG in the compositions advantageously provides synergistic antioxidant activity and excellent anti-aging and skin brightening benefits.

### SUMMARY OF THE INVENTION

The present invention provides a topical composition comprising at least 10% by weight of ascorbic acid, about 0.05 to about 2.5% by weight of epigallocatechin gallate, water and 1,3-propanediol, wherein the weight ratio of water to 1,3-propanediol in the composition is at least 0.6.

The present invention further provides a topical composition comprising at least 15% by weight of ascorbic acid, about 0.1% by weight epigallocatechin gallate, feverfew extract, gluconolactone, water, and at least 40% by weight of 1,3-propanediol, wherein the weight ratio of water to 1,3-propanediol in the composition is at least 0.6.

The present invention further provides cosmetic methods for treating skin, comprising topically applying to skin in need of treatment for skin brightening or lightening these topical compositions.

### DETAILED DESCRIPTION OF THE INVENTION

Unless defined otherwise, all technical and scientific terms used herein have the meaning commonly understood by one of ordinary skill in the art to which the invention pertains. All publications, patent applications, patents, and other references mentioned herein are incorporated by reference.

As used herein, “topically applying” means directly laying on or spreading on outer skin, the scalp, or hair, e.g., by use of the hands or an applicator such as a wipe, roller, or spray.

As used herein, “cosmetically acceptable” means the ingredients the term describes are suitable for use in contact with tissues (e.g., the skin or hair) without undue toxicity, incompatibility, instability, irritation, allergic response, or the like.

As used herein, a “cosmetically acceptable active agent” is a compound (synthetic or natural) that has a cosmetic or therapeutic effect on the skin or hair.

Compositions of the present invention are suitable for treating signs of skin aging. As used herein, “signs of skin aging” includes the presence of lines including fine lines and wrinkles, loss of elasticity, uneven skin, blotchiness, diminished skin thickness, and abnormal or diminished synthesis of collagen, glycosaminoglycans, proteoglycans, elastin, or glycoproteins including fibronectin. In one embodiment, the sign of aging is selected from the presence of lines, fine lines, wrinkles, loss of elasticity, and abnormal or diminished synthesis of collagen, glycosaminoglycans, proteoglycans, elastin, or glycoproteins including fibronectin.

Compositions of the invention are also suitable for treating skin in need of brightening or lightening treatment. As used herein, the terms “brightening” or “lightening” the skin refers generally to lightening, brightening, whitening, and/or evening of the skin tone, skin color, and/or shade of skin, and/or to the reduction in sallowness, and/or to the lightening and/or fading of hyperpigmented marks and/or lesions including, but not limited to, pigmented spots, melanin spots, age spots, sun spots, senile lentigos, freckles, lentigos simplex, pigmented solar keratosis, seborrhoeic keratosis, melasma, acne marks, post-inflammatory hyperpigmentation, lentigines, ephelides, combinations of two or more thereof and the like. “Brightening” or “lightening” the skin also refers to increased skin radiance, glow, translucency and/or luminescence and/or obtaining a more radiant, glowing, translucent or luminous skin tone appearance or a less yellow or sallow skin tone. In certain preferred embodiments, “brightening” or “lightening” the skin refers to lightening and evening the skin tone, increasing skin radiance and/or lightening age spots. In certain other preferred embodiments, the present invention is directed to compositions and methods for use on skin in need of skin brightening or lightening treatment selected from sallow and/or darkened skin. In certain other preferred embodiments, the present invention is directed to compositions and methods for use on skin in need of skin brightening or lightening treatment selected from the group consisting of age spots, freckles, marks left after acne, and combinations of two or more thereof.

As used herein, “treatment” or “treating” means the amelioration, prophylaxis, or reversal of a condition, disease, or disorder, or at least one discernible symptom thereof. In one embodiment, “treatment” or “treating” refers to an amelioration, prophylaxis, or reversal of at least one measurable physical parameter related to the condition, disease, or disorder being treated, not necessarily discernible in or by the subject being treated. In another embodiment, “treatment” or “treating” refers to inhibiting or slowing the progression of a condition, disease, or disorder, either physically, e.g., stabilization of a discernible symptom, physiologically, e.g., stabilization of a physical parameter, or both. In another embodiment, “treatment” or “treating” refers to delaying the onset of a condition, disease, or disorder.

In certain embodiments, a composition of the invention is administered as a preventative measure. As used herein, “prevention” or “preventing” refers to a reduction of the risk of acquiring a given condition, disease, or disorder.

More broadly, the compositions of the invention may also be used to treat or prevent cosmetic, dermatological, or other conditions and disorders including, but not limited to, infections, deranged or disordered cutaneous or mucocutaneous tissue relevant to skin, nail and hair; oral, vaginal and anal mucosa; disturbed keratinization; inflammation; changes associated with intrinsic and extrinsic aging, and others which may or may not be related to cutaneous system. The manifestations include, but are not limited to, oily skin; acne; rosacea; age spots; blemished skin; blotches; cellulite; dermatoses; dermatitis; skin, nail and hair infections; dandruff; dryness or looseness of skin, nail and hair; xerosis; inflammation, or eczema; elastosis; herpes; hyperkeratosis; hyperpigmented skin; ichthyosis; keratoses; lentigines; melasmas; mottled skin; pseudofolliculitis barbae; photoaging and photodamage; pruritus; psoriasis; skin lines; stretch marks; thinning of skin, nail plate and hair; warts; wrinkles; oral or gum disease; irritated, inflamed, red, unhealthy, damaged or abnormal mucosa, skin, hair, nail, nostril, ear canal, anal or vaginal conditions; breakdown, defective synthesis or repair of dermal components; abnormal or diminished synthesis of collagen, glycosaminoglycans, proteoglycans and elastin, as well as diminished levels of such components in the dermis; uneven skin tone; uneven and rough surface of skin, nail and hair; loss or reduction of skin, nail and hair resiliency, elasticity and recoilability; laxity; lack of skin, nail and hair lubricants and luster; fragility and splitting of nail and hair; yellowing skin; reactive, irritating or telangiectatic skin; and dull and older-looking skin, nail and hair. In addition, the compositions of the current invention can be used for general care of skin, nail and hair; to improve skin texture and pores, flakiness and redness; to make skin soft, smooth, fresh, balanced, visibly clear, even-toned and brighter; to increase skin fullness and plumpness; and for skin bleach and lightening and wound healing; to reduce or prevent sweating or perspiration of underarm, crotch, palm, or other parts of the body.

As used herein, the term “subject” means any animal, preferably a mammal, most preferably a human, to whom a composition of the invention will be or has been administered. The term “mammal” as used herein, encompasses any mammal. Examples of mammals include, but are not limited to, cows, horses, sheep, pigs, cats, dogs, mice, rats, rabbits, guinea pigs, monkeys, and humans. In a preferred embodiment, the subject is a human.

As used herein, “wrinkle” includes fine lines, fine wrinkles, or coarse wrinkles. Examples of wrinkles include, but are not limited to, fine lines around the eyes (e.g., “crow's feet”), forehead and cheek wrinkles, frown-lines, and laugh-lines around the mouth.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

As used herein, “cosmetic” refers to beautifying or preserving, restoring, bestowing, simulating, or enhancing the appearance of bodily beauty or appearing to enhance beauty or youthfulness, specifically as it relates to the appearance of tissue or skin.

As used herein, “cosmetically effective amount” means an amount sufficient for treating or preventing one or more signs of skin aging, but low enough to avoid serious side effects. The cosmetically effective amount of the compound or composition will vary with the particular condition being treated, the age and physical condition of the end user, the severity of the condition being treated/prevented, the duration of the treatment, the nature of other treatments, the specific compound or product/composition employed, the particular cosmetically-acceptable carrier utilized, and like factors.

As used herein, “substantially free of” means the ingredient referred to is not directly or intentionally added to the formula. Preferably, “substantially free of” means containing less than about 1% of an ingredient. More preferably “substantially free of” means containing less than about 0.5% of an ingredient. Even more preferably “substantially free of” means containing less than about 0.1% by weight of an ingredient. The present compositions may be completely free of an ingredient, i.e., contain none of the ingredient.

Unless otherwise indicated, a percentage or concentration refers to a percentage or concentration by weight (i.e., % (W/W). A weight percent of an ingredient is based on the total weight of the composition containing the ingredient. Unless stated otherwise, all ranges are inclusive of the endpoints, e.g., “from 4 to 9” includes the endpoints 4 and 9.

### Ascorbic Acid

The composition comprises ascorbic acid, also known as Vitamin C. Ascorbic acid has the following structure:

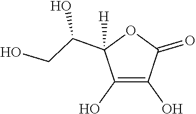 [SMILES: O=C1O[C@H]([C@@H](O)CO)C(O)=C1O]

The composition contains an unexpectedly high level of Vitamin C. The composition contains at least 10% by weight of ascorbic acid based on the total weight of the composition. The composition may contain at least 15% by weight of ascorbic acid based on the total weight of the composition.

The composition may contain an unexpectedly high level of Vitamin C that remains stable over time. As known in the art, compositions containing Vitamin C are preferably prepared with an overage of the ingredient to account for some degradation during the product's shelf life.

### Epigallocatechin Gallate (EGCG)

The composition also contains EGCG. The amount of EGCG in the composition may range from about 0.05 to about 2.5% by weight based on the total weight of the composition.

Preferably, the composition contains about 0.1% by weight of EGCG based on the total weight of the composition.

### Aqueous Vehicle

The composition contains an aqueous vehicle comprising water and 1,3-propanediol. Preferably, water and 1,3-propanediol are the only solvents in the composition.

The weight ratio of water:1,3-propanediol is at least 0.6. This vehicle unexpectedly provides excellent ascorbic acid stability in the composition over time. The weight ratio of water:1,3-propanediol may be at least 0.8.

The amount of water in the composition ranges from about 20 to about 40% by weight of the composition based on the total weight of the composition. Preferably, the amount of water in the composition is about 30% by weight of the composition based on the total weight of the composition.

The amount of 1,3-propanediol in the composition is preferably at least 40% by weight of the composition based on the total weight of the composition. More preferably, the amount of 1,3-propanediol in the composition is at least 45% by weight of the composition based on the total weight of the composition.

### Other Cosmetically Acceptable Active Agents

The composition may contain one or more other cosmetically acceptable active agents.

Cosmetically acceptable active agents include for example other anti-aging agents, other antioxidants, anti-acne agents, shine control agents, anti-microbial agents, anti-inflammatory agents, anti-mycotic agents, anti-parasite agents, external analgesics, sunscreens, photoprotectors, keratolytic agents, surfactants, moisturizers, nutrients, other vitamins, energy enhancers, anti-perspiration agents, astringents, deodorants, firming agents, anti-callous agents, and agents for hair and/or skin conditioning

The amount of other cosmetically active agent in the composition may range from about 0.001% to about 20% by weight of the composition, e.g., about 0.005% to about 10% by weight of the composition, such as about 0.01% to about 5% by weight of the composition, based on the total weight of the composition.

The cosmetically acceptable active agent may be selected for instance from alpha and polyhydroxy acids such as glycolic acid, lactic acid, malic acid, salicylic acid, citric acid, tartaric acid, and gluconolactone, benzoyl peroxide, D-panthenol carotenoids, retinoids such as retinol and retinyl palmitate, ceramides, polyunsaturated fatty acids, essential fatty acids, enzymes such as laccase, enzyme inhibitors, minerals, hormones such as estrogens, steroids such as hydrocortisone, 2-dimethylaminoethanol, copper salts such as copper chloride, peptides such as dipeptides including N-acyl dipeptide derivatives, tripeptides, argireline and syn-ake, those containing copper, coenzyme Q10, amino acids such as proline, other vitamins, lactobionic acid, acetyl-coenzyme A, niacin, riboflavin, thiamin, ribose, electron transporters such as NADH and FADH2, natural extracts such as those from Moringa Oleifera seed, aloe vera, feverfew (for example, Chrysanthemum Parthenium (Feverfew) Flower/Leaf/Stem Juice), oatmeal, dill, blackberry, princess tree, lemon aspen, resorcinols such as 4-hexyl resorcinol, curcuminoids, sugar amines such as N-acetyl glucosamine, and derivatives and mixtures thereof.

Examples of other vitamins include, but are not limited to, vitamin A, vitamin B's such as vitamin B3, vitamin B5, and vitamin B12, vitamin K, and different forms of vitamin E such as alpha, beta, gamma or delta tocopherols or their mixtures, and derivatives thereof.

Examples of other antioxidants include, but are not limited to, water-soluble antioxidants such as sulfhydryl compounds and their derivatives (e.g., sodium metabisulfite and N-acyl-cysteine), lipoic acid and dihydrolipoic acid, resveratrol, lactoferrin, and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, pine bark, and propolis.

The composition for example may comprise ascorbic acid, EGCG, and Moringa Oleifera seed extract. Such a composition may comprise at least 10% by weight of ascorbic acid and about 0.1% by weight of EGCG. Preferably, such a composition also comprises about 0.5% by weight of Moringa Oleifera seed extract.

The composition may preferably comprise ascorbic acid, EGCG, feverfew extract, and gluconolactone. Such a composition may comprise at least 15 weight percent ascorbic acid and about 0.1 weight percent EGCG. Preferably, such a composition also comprises about 1 weight percent feverfew extract and about 2 weight percent gluconolactone.

### Topical Compositions

The compositions of the present invention are applied topically to skin or hair, preferably skin. They are single phase aqueous compositions.

The compositions may be made into a wide variety of liquid product types that include but are not limited to solutions, lotions, creams, gels, sprays, ointments, and washes. The composition is preferably in the form of a serum, which is a liquid that is light and easily spread on and absorbed by the skin.

The composition may contain one or more emollients as known in the art. As used herein, “emollients” refer to materials used for the prevention or relief of dryness, such as by preventing the transepidermal loss of water from the skin. Examples of emollients include, but are not limited to, those set forth in the International Cosmetic Ingredient Dictionary and Handbook, eds. Pepe, Wenninger and McEwen, pp. 2930-36 (The Cosmetic, Toiletry, and Fragrance Assoc., Washington, D.C., 9th Edition, 2002) (hereinafter “ICI Handbook”). Examples of particularly suitable emollients include vegetable oils, mineral oils, fatty esters, and the like.

The composition can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contain between about 0.1% and 5%, by weight, of such gelling agents.

The compositions may contain, in addition to the above components, a wide variety of additional oil-soluble materials and/or water-soluble materials conventionally used in compositions for use on skin and hair, at their art-established levels. These include humectants, pH adjusters, fragrances, dyes, and preservatives (e.g., parabens).

The composition may comprise one or more oils. As used herein, the term “oil” means a hydrophobic material, for example hydrocarbon-based oils, silicones, fatty acid derivatives, glycerides, vegetable oils, vegetable oil derivatives, alkyl esters, wax esters, beeswax derivatives, sterols, and phospholipids.

Suitable hydrocarbon oils include petrolatum, mineral oil, micro-crystalline waxes, squalene and combinations thereof.

Silicone oils include dimethicone, dimethiconol, phenyl dimethicone and cyclic polysiloxanes and combinations thereof. Silicone oils having viscosities from about 0.5 to about 100,000 centistokes at 25° C. may also be useful in the composition.

Glycerides include castor oil, sunflower seed oil, coconut oil and derivatives, vegetable oils and derivatives, palm oil, jojoba oil, Shea butter, lanolin and combinations thereof.

Alkyl ester oils include, but are not limited to, isopropyl esters of fatty acids and esters of long chain fatty acids. More preferably, the following alkyl esters are useful: isopropyl palmitate, isopropyl myristate, myristyl myristate, isohexyl palmitate, decyl oleate, isononyl isononanoate and combinations thereof.

Preferably, the composition contains caprylyl glycol. More preferably the composition contains at least about 0.25% by weight of caprylyl glycol based on the total weight of the composition. For example, the composition may comprise at least about 0.5% by weight of caprylyl glycol based on the total weight of the composition.

Optionally, the composition may be substantially free of phenoxyethanol.

### pH

In one embodiment, the topical composition has a pH of about 3.6 to about 4.2 For example, the pH may be about 3.8.

The topical composition may comprise a buffering agent such as lactic acid, citric acid, malic acid, tartaric acid, gluconic acid, or gluconolactone to maintain the pH. Typically, the composition contains about 2 to about 12, or about 4 to about 8, weight percent of buffering agent.

### Methods of Treating Skin

According to the invention, skin in need of treatment for skin brightening or lightening may be treated by topically applying a composition comprising at least 10% by weight of ascorbic acid, about 0.05 to about 2.5% by weight of epigallocatechin gallate, water and 1,3-propanediol, wherein the weight ratio of water to 1,3-propanediol in the composition is at least 0.6.

Also according to the invention, skin in need of treatment for skin brightening or lightening may be treated by topically applying a composition comprising at least 15% by weight of ascorbic acid based on the total weight of the composition, about 0.1% by weight epigallocatechin gallate based on the total weight of the composition, feverfew extract, gluconolactone, water, and at least 40% by weight of 1,3-propanediol based on the total weight of the composition, wherein the weight ratio of water to 1,3-propanediol in the composition is at least 0.6.

Compositions according to the invention comprising at least 10% by weight of ascorbic acid, EGCG, water and 1,3-propanediol, wherein the weight ratio of water to 1,3-propanediol in the composition is at least 0.6, also advantageously and surprisingly provide superior ascorbic acid solubility and stability.

The following non-limiting examples further illustrate the invention.

### Example 1

Three compositions, each containing 17% by weight ascorbic acid, 0.1% by weight EGCG, 2% by weight gluconolactone, and 1% by weight feverfew in water/1,3-propanediol vehicles were tested for stability. The compositions had different weight ratios of water to 1,3-propanediol, as shown below in Table 1. However, the combined total amount of water and 1,3-propanediol was the same in all the compositions, 77.25% by weight.

The compositions were tested for ascorbic acid solubility by evaluating visually for at 4° C. and noting by the absence or presence of ascorbic acid crystals.

The results are also shown in Table 1.

TABLE 1

| | Composition 1 | Composition 2 | Composition 3 |
|---|---|---|---|
| Purified Water | 25.25% | 29.55% | 33.45% |
| 1,3-Propanediol | 52.00% | 47.70% | 43.80% |
| Ascorbic Acid | 17.00% | 17.00% | 17.00% |
| Gluconolactone | 2.00% | 2.00% | 2.00% |
| Epigallocatechin | 0.10% | 0.10% | 0.10% |
| Gallate | | | |
| Feverfew Extract | 1.00% | 1.00% | 1.00% |
| Sodium Hydroxide, | 2.65 (QS) | 2.65 (QS) | 2.65 (QS) |
| 10% (w/w) | | | |
| Aqueous Solution | | | |
| 4 C. 1 Month 1 | Many Crystals | One Crystal | Clear |
| Week (Cold) | | | |
| 4 C. 1 Month 1 | Many Crystals | Clear | Clear |
| Week (25 C.) | | | |
| 4 C. 1 Month 3 | Many Crystals | Clear | Many Crystals |
| Weeks (Cold) | | | |
| 4 C. 2 Months (25 | Many Crystals | Clear | Clear |
| C.) | | | |
| 4 C. 2 Months 1 | Many Crystals | Clear | Clear |
| Week (Cold) | | | |
| Water:1,3-Propanediol | 0.4856 | 0.6195 | 0.7637 |
| Weight Ratio | | | |

These results show that compositions containing ascorbic acid and EGCG and having a weight ratio of water to 1,3-propanediol of about 0.6 or higher according to the invention had superior stability to compositions containing the same ingredients but having a weight ratio of water to 1,3-propanediol of less than 0.6, even when containing the same total amount of water and 1,3-propanediol. Moreover, surprisingly, Composition 2 containing an intermediate amount of water (29.55% by weight) and having an intermediate weight ratio of water to 1,3-propanediol (0.6195) provided the best ascorbic acid stability of the three compositions.

### Example 2

Three compositions each containing 17% by weight ascorbic acid, 0.1% by weight EGCG, and 1% by weight feverfew in water/1,3-propanediol vehicles were tested for stability. The compositions contained different amounts of gluconolactone (2%, 0.5% and 0% weight percent) as shown in Table 2.

The compositions were prepared in the same way as described in Example 1.

The compositions were tested for ascorbic acid stability as follows. The compositions were monitored at 25° C./60% Relative Humidity (RH) and 40° C./75% RH. Samples were measured by HPLC analysis for the concentration of ascorbic acid at initial and several timepoints over 12 weeks.

The results are shown in Table 2.

TABLE 2

| | Composition 4 | Composition 5 | Composition 6 |
|---|---|---|---|
| Purified Water | 29.55% | 29.55% | 29.55% |
| 1,3-Propanediol | 47.70% | 49.20% | 49.7% |
| Ascorbic Acid | 17.00% | 17.00% | 17.00% |
| Gluconolactone | 2.00% | 0.50% | 0.00% |
| Epigallocatechin | 0.10% | 0.10% | 0.10% |
| Gallate | | | |
| Feverfew Extract | 1.00% | 1.00% | 1.00% |
| Sodium Hydroxide, | 2.65 (QS) | 2.65 (QS) | 2.65 (QS) |
| 10% (w/w) Aqueous | | | |
| Solution | | | |
| Initial Vitamin C | 16.97% | 17.05% | 17.02% |
| Content | | | |
| 1 Month 40 C. Assay | 15.48% | 15.36% | 13.61% |
| 2 Months 40 C. Assay | 14.37% | 13.55% | |
| Water:1,3-Propanediol | 0.6195 | 0.6006 | 0.5946 |
| Weight Ratio | | | |

These results surprisingly show ascorbic acid stability increased with increasing amounts of gluconolactone in the formulation. Composition 5, both containing 2% by weight gluconolactone and having a water:1,3-propanediol weight ratio of 0.6195 had the greatest ascorbic acid stability of all three compositions.

### Example 3

The combined anti-oxidant effects of combinations of ascorbic acid with various other known anti-oxidants was tested using the oxygen radical absorbance capacity (ORAC) assay according to the method described by Ou B, Hampsch Woodill M, Prior R. L. Development and validation of an improved oxygen radical absorbance capacity assay using fluorescein as the fluorescent probe. J Agric Food Chem. 2001, 49:4619 (using a slightly different ex/em wavelength readout and a narrower range of Trolox standards). This assay measures the ability of anti-oxidant “chain breakers” to inhibit the decline in disodium fluorescein (FL) fluorescence that is induced by the peroxyl radical generator, 2′,2′-Azobis (2-amidinopropane) dihydrochloride (AAPH). Distilled water was used as the negative control. Trolox (6-hydroxy-2,5,7,8-tetramethylchroman-2-carboxylic acid, a water-soluble analog of vitamin E sold by Hoffman-LaRoche having antioxidant properties) was used as the positive control.

The final ORAC values were calculated from the regression equation of net areas under the FL decay curve using a formula where Area Under the Curve (AUC)=1+(Point 1+Point 2+Point 3+ . . . +Point 15)/Point 1. The Trolox standards were then plotted as micromoles over AUC. The samples were solved using their AUC yielding the values in micromole (μmol) Trolox equivalent (TE) per ml. Applied BioSystems Cytofluor 4000 fluorometer & software was used for quantification. ORAC values of the test materials were expressed in μmoles TE/ml (TE=Trolox Equivalents).

All ingredients were stored at room temperature and then diluted with distilled water into aqueous stock solutions for testing. Further dilutions were made in distilled water within 24 h from testing.

The results are shown in Tables 3 and 4, which report two separate test runs.

TABLE 3

| | | Expected | |
|---|---|---|---|
| | ORAC | (Additive) | |
| | (μmoles | ORAC | |
| Test Material | TE/ml) | value | Synergy |
| Water (Negative Control) | 0 | | |
| 10% ascorbic acid | 751 | | |
| 0.5% Aqua (and) Glycerin (and) | 12 | | |
| Avena Sativa (Oat) Kernel | | | |
| Extract | | | |
| 2% caffeine | 10 | | |
| 0.5% Ethyl L-Ascorbic Acid | 13 | | |
| 0.5% sodium ascorbyl phosphate | 10 | | |
| 0.5% Tetrahydrodiferuloyl-methane | 4 | | |
| 0.01% Mannitol (and) Ergothioneine | 0 | | |
| 0.1% Ergothioneine (and) Tricholoma | 0 | | |
| Matsutake Mycelium Ferment | | | |
| Extract) | | | |
| 0.5% Ergothioneine (and) Tricholoma | 1.5 | | |
| Matsutake Mycelium Ferment | | | |
| Extract) | | | |
| 0.5% Chrysanthemum Parthenium | 4 | | |
| (Feverfew) Extract | | | |
| 0.1% EGCG | 7 | | |
| 10% Vit C + 0.5% Aqua (and) | 556 | 763 | No |
| Glycerin (and) Avena Sativa | | | |
| (Oat) Kernel Extract | | | |
| 10% Vit C + 2% caffeine | 1111 | 761 | Yes |
| 10% Vit C + 0.5% Ethyl L-Ascorbic | 924 | 764 | Yes |
| Acid | | | |
| 10% Vit C + 0.5% sodium ascorbyl | 697 | 761 | No |
| phosphate | | | |
| 10% Vit C + 0.5% | 263 | 755 | No |
| Tetrahydrodiferuloyl-methane | | | |
| 10% Vit C + 0.01% Mannitol (and) | 628 | 751 | No |
| Ergothioneine | | | |
| 10% Vit C + 0.1% Ergothioneine | 602 | 751 | No |
| (and) Tricholoma Matsutake | | | |
| Mycelium Ferment Extract) | | | |
| 10% Vit C + 0.5% Ergothioneine | 598 | 752.2 | No |
| (and) Tricholoma Matsutake | | | |
| Mycelium Ferment Extract) | | | |
| 10% Vit C + 0.5% Chrysanthemum | 638 | 755 | No |
| Parthenium (Feverfew) Extract | | | |
| 10% Vit C + 0.1% EGCG | 859 | 758 | Yes |
| Trolox (Positive Control) | 3940 μmol/g | | |

TABLE 3

| | ORAC | Expected | |
|---|---|---|---|
| | (μmoles | (Additive) | |
| Test Material | TE/ml) | ORAC value | Synergy |
| Water (Negative Control) | 0 | | |
| 10% ascorbic acid | 838 | | |
| 1% caffeine | 0 | | |
| 0.1% Ethyl L-Ascorbic Acid | 7 | | |
| 10% Vit C + 1% Caffeine | 688 | 838 | No |
| 10% Vit C + 0.1% Ethyl | 891 | 845 | Yes |
| L-Ascorbic Acid | | | |
| Trolox (Positive Control) | 4368 μmol/g | | |

### Example 4

Two topical compositions according to the invention were made using the ingredients shown in Table 4.

TABLE 4

| | Composition 7 | Composition 8 |
|---|---|---|
| INCI Names | % w/w | % w/w |
| Water | 31.616 | 39.35 |
| 1,3-Propanediol | 46.95 | 47 |
| Disodium EDTA | 0 | 0.2 |
| Epigallocatechin Gallate | 0.1 | 0.1 |
| Phenoxyethanol | 0.5 | 0 |
| Caprylyl Glycol | 0.25 | 0.25 |
| Ascorbic Acid | 17 | 12.5 |
| Gluconolactone | 2 | 0 |
| Chrysanthemum | 1 | 0 |
| Parthenium (Feverfew) | | |
| Flower/Leaf/Stem Juice | | |
| Water; Glycerin, Moringa | 0 | 0.5 |
| Oleifera Seed Extract | | |
| Sodium Hydroxide | 0.584 | 0.1 |
| TOTAL | 100 | 100 |
| Water:1,3-Propanediol | 0.6734 | 0.8372 |

### Example 5

A 12-week, Institutional Review Board approved, single center, prospective clinical study with direct comparison to baseline condition was performed using Composition 7 according to the invention (Table 4).

The study population was females, ages 30 to 60 years with Fitzpatrick Skin Types I-VI. Key inclusion criteria were moderate score for lack of clarity and radiance and mild to moderate uneven skin tone on the global face (modified Griffith's scale); textural parameters such as fine lines and wrinkles were included as appropriate to ensure inclusion of all skin tones. 44 racially and ethnically diverse women representing all Fitzpatrick Skin Types (I-VI) completed the study; 43% Black/African American, 18% Asian, 37% Caucasian, 2% Multi racial with 9% being Hispanic/Latino.

Clinical grading for pigmentation and textural attributes and tolerability were collected along with self-assessment questionnaires and digital photography. Clinical grading scores were compared to baseline scores for each subject at each visit using a Wilcoxon signed rank test, p<0.05. Percent changes from baseline were calculated from mean delta scores. Self-assessment scores were tabulated.

Composition 7 was applied once daily in the morning. Bland day SPF 35 and night moisturizers were provided to standardize the regimen.

All 12 clinically graded parameters (hyperpigmentation, evenness of skin tone, clarity, radiance/luminosity/brightness, fine lines, wrinkles, pore size, laxity, global lift, tactile roughness/smoothness, visual roughness/smoothness, and overall photodamage/appearance) showed significant improvement at week 12, p<0.05.

Eight of the 12 parameters (fine lines, wrinkles, pore size, laxity, global lift, tactile roughness/smoothness, visual roughness/smoothness, and overall photodamage/appearance) significantly improved at week 4 and continued through week 8 and week 12, p≤0.05. This included all four pigmentation/brightening parameters: hyperpigmentation, evenness of skin tone, clarity, and radiance/luminosity/brightness.

The self-assessment results were as follows:
Early benefits noticed after 1 week
   98% brighter complexion, more radiant
   100% smoother texture
Multiple benefits to hyperpigmentation and texture After 12 weeks
   100% skin is more brilliant
   95% complexion is dramatically brighter
   95% skin feels protected against environmental stressors
   95% fine lines are less visible
   93% skin tone is dramatically more even
   90% dark spots are less apparent

Accordingly, Composition 7 according to the invention advantageously delivered clinically and consumer-perceivable benefits for overall facial skin brightening in a diverse population representing all skin tones.

1. A topical composition comprising at least 10% by weight of ascorbic acid, about 0.05 to about 2.5% by weight of epigallocatechin gallate, water and 1,3-propanediol, wherein the weight ratio of water to 1,3-propanediol in the composition is at least 0.6.

2. The composition of claim 1 comprising about 0.1% by weight of epigallocatechin gallate based on the total weight of the composition.

3. The composition of claim 1 comprising at least 40% by weight of 1,3-propanediol based on the total weight of the composition.

4. The topical composition of claim 1, wherein the weight ratio of water to 1,3-propanediol in the composition is at least 0.8.

5. The composition of claim 1 having a pH of about 3.6 to about 4.2.

6. The composition of claim 1 in the form of a single aqueous phase.

7. A topical composition comprising at least 15% by weight of ascorbic acid based on the total weight of the composition, about 0.1% by weight epigallocatechin gallate based on the total weight of the composition, feverfew extract, gluconolactone, water, and at least 40% by weight of 1,3-propanediol based on the total weight of the composition, wherein the weight ratio of water to 1,3-propanediol in the composition is at least 0.6.

8. A cosmetic method for treating skin, comprising topically applying to skin in need of treatment for skin brightening or lightening the topical composition of claim 1.
